(19) United States
(12) Patent Application Publication
(10) Pub. No.: US 2025/0283963 A1
(43) Pub. Date: Sep. 11, 2025

(54) Multi-Spectral Susceptibility-Weighted Magnetic Resonance Imaging

(71) Applicant: The Medical College of Wisconsin, Inc., Milwaukee, WI (US)

(72) Inventors: Kevin Matthew Koch, Brookfield, WI (US); Andrew Scott Nencka, Franklin, WI (US)

(21) Appl. No.: 18/859,018

(22) PCT Filed: Apr. 24, 2023
(86) PCT No.: PCT/US2023/019570

§ 371 Date: Oct. 22, 2024

Related U.S. Application Data
(60) Provisional application No. 63/333,885, filed on Apr. 22, 2022.

(51) Int. Cl. G01R 33/56; A61B 5/00; A61B 5/055; G01R 33/44; G01R 33/50

(52) U.S. Cl. CPC G01R 33/5608; A61B 5/0042; A61B 5/055; G01R 33/445; G01R 33/50

(57) ABSTRACT

Images with susceptibility weighted image contrast are generated from data acquired using a multispectral magnetic resonance imaging (“MSI”) acquisition. The inherent spectral information within multispectral imaging data are used to generate spectral perturbation maps that approximate phase contrast maps, from which images with susceptibility weighted image contrasts can be generated. The resulting images may have suppressed or otherwise reduced metal artifacts (i.e., artifacts generated by or otherwise associated with metallic objects), and/or may be reconstructed from data acquired using a magnetic resonance imaging (“MRI”) system with significant B₀ field inhomogeneities, such as a low-field MRI system with a B₀ field strength less than 0.3 T.

### CROSS-REFERENCE TO RELATED APPLICATIONS

This application claims the benefit of U.S. Provisional Patent Application Ser. No. 63/333,885, filed on Apr. 22, 2022, and entitled “MULTI-SPECTRAL SUSCEPTIBILITY-WEIGHTED MAGNETIC RESONANCE IMAGING IN THE PRESENCE OF METALLIC HARDWARE,” which is herein incorporated by reference in its entirety.

### STATEMENT OF FEDERALLY SPONSORED RESEARCH

This invention was made with government support under EB030123 awarded by the National Institutes of Health. The government has certain rights in the invention.

### BACKGROUND

When performing magnetic resonance imaging (“MRI”) in the presence of metal objects, such as surgical implants or other metal containing medical devices, significant image artifacts are often induced by the metal object(s), thereby rendering the images non-diagnostic. Commonly encountered metal objects in neuroimaging include aneurysm clips, flow diverters, ventriculoperitoneal shunts, cochlear implants, and permanent dental/orthodontic hardware.

Multispectral imaging sequences significantly reduce the dominant MRI artifacts around metal objects. To date, the image contrasts available using MSI have been developed for exclusively orthopedic applications (i.e., imaging near total joint replacements). Although T1, T2, and diffusion-weighted MSI sequences have been developed, susceptibility-weighted imaging (“SWI”), which is a well-established diagnostic tool, is a missing contrast in the MSI neuroimaging portfolio.

Developing SWI contrast near metal implants is a significant challenge, as SWI utilizes T2* signal dephasing and in-plane phase accumulation to build its unique tissue contrast. T2* signal dephasing, which is enabled using gradient-echo pulse-sequences, is an image artifact induced by metal implants. As a result, gradient-echo sequences (and SWI by extension) are typically avoided whenever metallic instrumentation is near the imaging field of view (“FOV”) or otherwise within the bore of the MRI system.

### SUMMARY OF THE DISCLOSURE

The present disclosure addresses the aforementioned drawbacks by providing a method for magnetic resonance imaging, in which susceptibility weighted images are generated from multispectral imaging data. The method includes accessing multispectral imaging data with a computer system, where the multispectral imaging data comprise magnetic resonance data acquired using multiple different spectral offsets. A spectral off-resonance map is generated from the multispectral imaging data using the computer system, and an image with a susceptibility weighted image contrast is generated by multiplying a magnitude image from the multispectral imaging data with the spectral off-resonance map.

It is another aspect of the present disclosure to provide a method for magnetic resonance imaging, in which susceptibility weighted images are generated from multispectral imaging data. The method includes accessing multispectral imaging data with a computer system, where the multispectral imaging data comprise magnetic resonance data acquired using multiple different spectral offsets; accessing spectral off-resonance data with the computer system, where the spectral off-resonance data are generated from the multispectral imaging data; and accessing a neural network with the computer system, where the neural network has been trained on training data to generate images with susceptibility weighted image contrast based on inputs of multispectral imaging data and spectral off-resonance data. An image with a susceptibility weighted image contrast is generated by inputting the multispectral imaging data and the spectral off-resonance data to the neural network using the computer system, generating output as the image with the susceptibility weighted image contrast.

The foregoing and other aspects and advantages of the present disclosure will appear from the following description. In the description, reference is made to the accompanying drawings that form a part hereof, and in which there is shown by way of illustration one or more embodiments. These embodiments do not necessarily represent the full scope of the invention, however, and reference is therefore made to the claims and herein for interpreting the scope of the invention.

### BRIEF DESCRIPTION OF THE DRAWINGS

FIG. 1 is a flowchart of an example method for generating susceptibility weighted images from multispectral magnetic resonance imaging data.

FIG. 2 illustrates a comparison of field maps generated using 3D-MSI techniques (left column) and susceptibility weighted images generated using conventional SWI processing techniques (right column).

FIG. 3 is a flowchart of an example method for generating susceptibility weighted images from multispectral magnetic resonance imaging data and spectral off-resonance data using a suitably trained neural network.

FIG. 4 is a flowchart of an example method for training a neural network to generate susceptibility weighted images from multispectral magnetic resonance imaging data and spectral off-resonance data.

FIGS. 5A-5G illustrate example susceptibility weighted images generated using the methods described in the present disclosure and comparisons with conventionally generated susceptibility weighted images.

FIGS. 6A-6K illustrate another example susceptibility weighted images generated using the methods described in the present disclosure and comparisons with conventionally generated susceptibility weighted images.

FIGS. 7A-7K illustrate another example susceptibility weighted images generated using the methods described in the present disclosure and comparisons with conventionally generated susceptibility weighted images.

FIG. 8 is a block diagram of an example magnetic resonance imaging (“MRI”) system that can implement the methods described in the present disclosure.

FIG. 9 is a block diagram of an example system for generating susceptibility weighted images from multispectral imaging data.

FIG. 10 is a block diagram of example components that can implement the system of FIG. 9.

### DETAILED DESCRIPTION

Described here systems and methods for generating magnetic resonance images with susceptibility weighted image contrast from data acquired using a multispectral acquisition technique. Advantageously, the resulting susceptibility weighted images have suppressed or otherwise reduced metal artifacts (i.e., artifacts generated by or otherwise associated with metallic objects). In general, the disclosed systems and methods generate susceptibility weighed imaging (“SWI”) contrasts using three-dimensional (“3D”) multispectral imaging (“MSI”) techniques. A 3D-MSI pulse sequence (e.g., a spin-echo based 3D-MSI pulse sequence) is used to acquire data, and the inherent spectral information within those MSI data are used to generate spectral perturbation maps that approximate phase contrast maps, from which images with SWI-like contrasts can be generated. In some examples, the acquisition of data using an MSI technique allows for the reduction of metal object induced artifacts.

Additionally or alternatively, the systems and methods described in the present disclosure can be used to generate images with SWI-like contrasts using low-field MRI systems, which may include MRI systems operating with a main magnetic field (B₀) strength that is less than or equal to approximately 0.3 T. In general, low-field MRI systems may be classified as a low-field MRI system, a very-low-field MRI system, or an ultra-low-field MRI system within the low-field regime. A “low-field” MRI system may have a B₀ field strength between 0.1-0.3 T, a “very-low-field” MRI system may have a B₀ field strength between 10 mT and 0.1 T, and an “ultra-low-field” MRI system may have a B field strength less than 10 mT. Thus, as used herein, a low-field MRI system may include a low-field MRI system, a very-low-field MRI system, and/or an ultra-low-field MRI system. These low-field MRI systems often have significant B₀ inhomogeneities relative to MRI systems with higher field strengths (e.g. those with 0.5 T, 1.5 T, 3 T, or greater).

Advantageously, the application of the disclosed systems and methods to these low-field MRI systems allows for acquiring images with SWI-like contrast, which are otherwise challenging to acquire or otherwise unattainable with these low-field MRI systems, by treating the inherent B₀ inhomogeneities of these low-field MRI systems as sources of off-resonances for an MSI data acquisition. Accordingly, the systems and methods allows for low-field MRI systems to provide clinical applications such as stroke screening (e.g., occlusive, rupture) based on the SWI-like contrast imaging. Additionally or alternatively, quantitative susceptibility mapping (“QSM”) techniques may also be performed on low-field MRI systems using the systems and methods described in the present disclosure. By enabling QSM techniques on these low-field MRI systems, clinical applications that utilize differentiating calcifications, blood products, and the like can also be achieved.

Advantageously, the disclosed systems and methods address the unmet needs for metal artifact suppressed SWI in neuroradiology. The metal-suppressed SWI approaches described in the present disclosure have potential applications in patient populations with high susceptibility intra-cranial hardware, such as aneurysm clips, vascular shunts, cochlear implants, and/or fixed dental/orthodontic hardware.

Conventional SWI techniques generate phase-accumulation maps from T2*-weighted gradient-echo (“GRE”) acquisitions, high-pass filter the phase maps, and then multiply the magnitude T2*-weighted images with the filtered phase maps raised to an exponential factor, generating output as susceptibility weighted images. It is a discovery of the present disclosure that spectral perturbation maps estimated from multispectral imaging data can be used as a reasonable approximation of the phase accumulation maps from a conventional SWI processing workflow. These spectral perturbation maps leverage magnitude spectral-bin image data to estimate local off-resonance values. As noted above, in some instances the sources of the spectral perturbations may be metallic hardware implanted in the subject being imaged. In some other instances, the sources of the spectral perturbations may be from B₀ inhomogeneities, such as those present in low-field MRI systems.

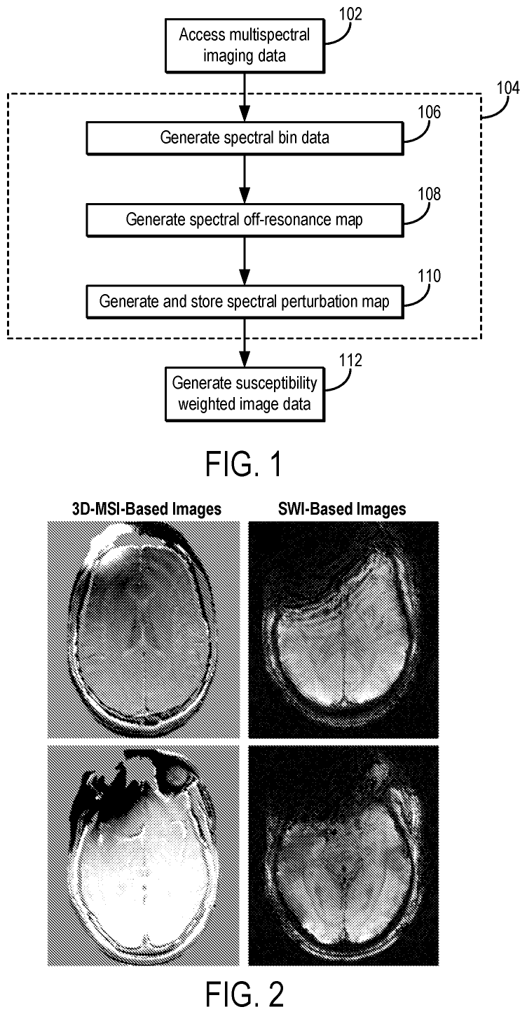

Referring now to FIG. 1, a flowchart is illustrated as setting forth the steps of an example method for estimating or otherwise generating susceptibility weighted images from data acquired using a multispectral imaging technique. As an advantage of using a multispectral imaging technique, the resulting susceptibility weighted images can have reduced artifacts associated with metal artifacts (e.g., reduced signal dropouts). Additionally or alternatively, using the multispectral imaging technique enables the acquisition of images with SWI-like contrasts with low-field MI systems.

The method includes accessing multispectral imaging data with a computer system, as indicated at step 102. Accessing the multispectral imaging data can include retrieving previously acquired data from a memory or other suitable data storage device or medium. Additionally or alternatively, accessing the multispectral imaging data can include acquiring such data with an MRI system and communicating or otherwise transferring the data to the computer system, which in some embodiments may be a part of the MRI system.

In general, the multispectral imaging data include magnetic resonance imaging data (e.g., k-space data, images reconstructed from k-space data) acquired using multispectral imaging (“MSI”) techniques. In an MSI acquisition, multiple images are acquired with incremented offsets of the transmit and/or receive frequencies, which may be referred to as different spectral bins. Spectral bin images can be reconstructed from the acquired data. The multispectral imaging data can be 2D data or 3D data. As a non-limiting example, the multispectral imaging data can be acquired using a 3D pulse sequence, such as a 3D gradient-echo pulse sequence, a 3D fast spin-echo pulse sequence, or the like.

Native T2* dephasing contrast is generally incompatible with imaging metallic instrumentation, so alternative magnitude contrast options are preferred. FIG. 2 demonstrates this problem, displaying the SWI artifact encountered when a high susceptibility object (cobalt-chromium implant) is placed on the skull within the imaging FOV. The 3D-MSI field maps (left column) show the presence of the implant, which substantially degrades the conventional SWI images (right column), eliminating the majority of the anterior portion of the brain signal. FIG. 2 shows two rows of slices showing 3D-MSI based field maps (left column) and conventional SWI images (right column) within the same slice for a test case with a cobalt-chromium implant in the field-of-view located at the top-left outside of the subject's eye socket. The impact on the SWI images is substantial, showing complete signal loss in most of the frontal lobe. Similar signal loss is observed when attempting to acquire SWI images using low-field MRI systems, or other MRI systems with significant B₀ inhomogeneities.

As non-limiting example, T1-weighted and intermediate-weighted 3D-MSI can be utilized for magnitude weighting. As noted, the multispectral imaging data may include k-space data acquired using an MSI technique, or may include images reconstructed from such k-space data. When the provided data include k-space data, images (e.g., spectral bin images) can be reconstructed by the computer system as part of step 102.

From the multispectral imaging data, spectral perturbation maps are generated using the computer system, as indicated generally at process block 104. As a non-limiting example, the spectral perturbation maps can be generated by extracting and saving intermediate spectral bin data (e.g., intermediate spectral bin magnitude data) from the multispectral imaging data, as indicated at step 106. A spectral off-resonance map is then computed from the spectral bin data, as indicated at step 108. In some implementations, the spectral off-resonance map can subsequently be denoised (e.g., using nonlocal means), or otherwise filtered (e.g., high-pass filtered). As a non-limiting example, the spectral off-resonance map can be denoised, high-pass filtered, and then raised to the fourth power. The spectral off-resonance map is then stored as the spectral perturbation map, as indicated at step 110.

The multispectral imaging data is then weighted using the spectral perturbation map(s) in order to generate one or more susceptibility weighted images, as indicated at step 112. For instance, the spectral perturbation map(s) can be multiplied by magnitude images in the multispectral imaging data (e.g., T1-weighted images), generating output as susceptibility weighted images. These susceptibility weighted images can be displayed to the user, or stored for later use. As a non-limiting example, the susceptibility weighted images can be further processed, such as by computing a minimum intensity projection, which can depict enhanced visualization of vessels within the brain.

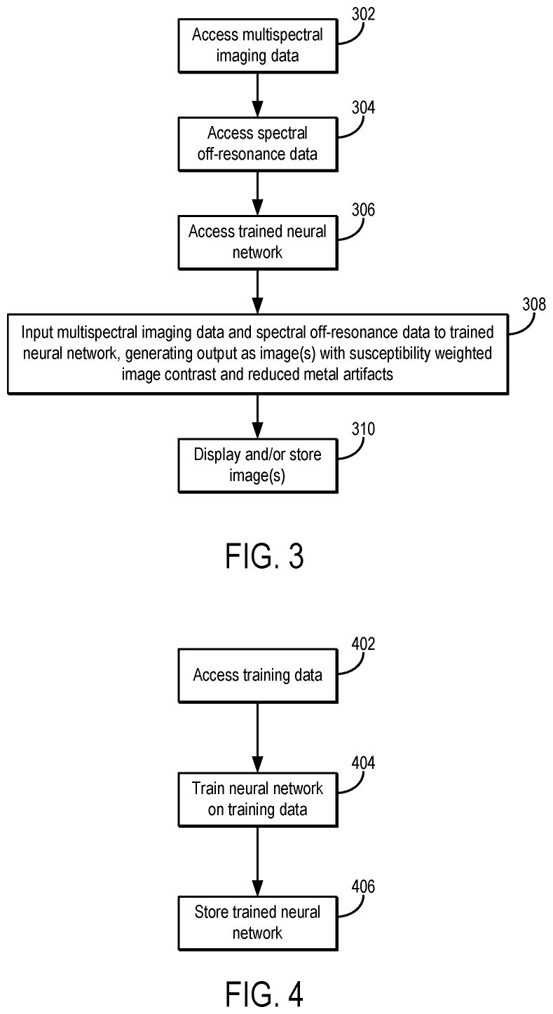

Referring now to FIG. 3, a flowchart is illustrated as setting forth the steps of an example method for generating susceptibility-weighted images from multispectral imaging data using a suitably trained neural network. As described above, by generating the susceptibility-weighted images from multispectral imaging data, metal artifacts can be reduced in the resulting images. Additionally or alternatively, the susceptibility-weighted images may be reconstructed from multispectral imaging data acquired using a low-field MRI system, or other MRI system having significant B₀ inhomogeneities. The method includes accessing multispectral imaging data with a computer system, as indicated at step 302. Accessing the multispectral imaging data can include retrieving previously acquired data from a memory or other suitable data storage device or medium. Additionally or alternatively, accessing the multispectral imaging data can include acquiring such data with an MRI system and communicating or otherwise transferring the data to the computer system, which in some embodiments may be a part of the MRI system. The method also includes accessing spectral off-resonance data with the computer system, as indicated at step 304. The spectral off-resonance data can include spectral perturbation maps, field maps, or the like, that are generated from multispectral imaging data. For example, the spectral off-resonance data can include spectral perturbation maps generated from the multispectral imaging data accessed in step 302. The spectral off-resonance data can include previously generated data, or can include be generated using the computer system after accessing the multispectral imaging data. In this way, accessing the spectral off-resonance data may include retrieving such data from a memory or other suitable data storage device or medium. Alternatively, accessing the spectral off-resonance data may include generating such data from multispectral imaging data.

A neural network is then accessed with the computer system, as indicated at step 306. Accessing the neural network may include accessing network parameters (e.g., weights, biases, or both) that have been optimized or otherwise estimated by training the neural network on training data. In some instances, retrieving the neural network can also include retrieving, constructing, or otherwise accessing the particular neural network architecture to be implemented. For instance, data pertaining to the layers in a neural network architecture (e.g., number of layers, type of layers, ordering of layers, connections between layers, hyperparameters for layers) may be retrieved, selected, constructed, or otherwise accessed.

In general, the neural network is trained, or has been trained, on training data in order to generate susceptibility weighted images from magnitude images in multispectral imaging data and spectral off-resonance maps. As a non-limiting example, the neural network can include a convolutional neural network (“CNN”). For instance, the CNN can be based on a V-Net architecture (e.g., for processing 3D datasets), a U-Net architecture (e.g., for processing 2D datasets), or the like.

The multispectral imaging data and the spectral off-resonance data are then input to the one or more trained neural networks, generating output as susceptibility weighted images, or as images that otherwise have a susceptibility weighted image contrast, as indicated at step 308. These susceptibility weighted images can be displayed to the user, or stored for later use, as indicated at step 310. As a non-limiting example, the susceptibility weighted images can be further processed, such as by computing a minimum intensity projection, which can depict enhanced visualization of vessels within the brain.

Referring now to FIG. 4, a flowchart is illustrated as setting forth the steps of an example method for training one or more neural networks on training data, such that the one or more neural networks are trained to receive input as spectral imaging data and spectral off-resonance data in order to generate output as susceptibility weighted images.

In general, the neural network(s) can implement any number of different model architectures or algorithm types. For instance, the neural network(s) could implement a convolutional neural network, a residual neural network, or other artificial neural network. In some instances, the neural network(s) may implement deep learning. Alternatively, the neural network(s) could be replaced with other suitable machine learning algorithms, such as those based on supervised learning, unsupervised learning, deep learning, ensemble learning, dimensionality reduction, and so on. As one non-limiting example, the neural network can include a convolutional neural network based on a V-Net architecture.

The method includes accessing training data with a computer system, as indicated at step 402. Accessing the training data may include retrieving such data from a memory or other suitable data storage device or medium. Alternatively, accessing the training data may include acquiring such data with an MRI system and transferring or otherwise communicating the data to the computer system, which may be a part of the MRI system. Additionally or alternatively, accessing the training data may include generating or otherwise assembling training data from magnetic resonance imaging data, multispectral imaging data, or the like.

Accessing the training data can, in some instances, include assembling training data from multispectral imaging data, spectral off-resonance data, and conventional susceptibility weighted images (e.g., conventional susceptibility weighted images acquired not in the presence of artifact-inducing metal objects, susceptibility-weighted images acquired with an MRI system other than a low-field MRI system) using a computer system. This step may include assembling the training data into an appropriate data structure on which the neural network can be trained. Assembling the training data may include assembling multispectral imaging data, spectral off-resonance data, and/or conventional susceptibility weighted images, and other relevant data. For instance, assembling the training data may include generating labeled data and including the labeled data in the training data. Labeled data may include multispectral imaging data, spectral off-resonance data, and/or conventional susceptibility weighted images that have been labeled as belonging to, or otherwise being associated with, one or more different classifications or categories. Creating labeled data may include labeling all data within a field-of-view of the data, or may include labeling only those data in one or more regions-of-interest within the data. The labeled data may include data that are classified on a voxel-by-voxel basis, or a regional or larger volume basis.

One or more neural networks are trained on the training data, as indicated at step 404. In general, the neural network(s) can be trained by optimizing network parameters (e.g., weights, biases, or both) based on minimizing a loss function. As one non-limiting example, the loss function may be a mean squared error loss function.

Training a neural network may include initializing the neural network, such as by computing, estimating, or otherwise selecting initial network parameters (e.g., weights, biases, or both). Training data can then be input to the initialized neural network, generating output data (e.g., intermediate susceptibility weighted image data). The quality of the output data can then be evaluated, such as by passing the output data to the loss function to compute an error. The current neural network can then be updated based on the calculated error (e.g., using backpropagation methods based on the calculated error). For instance, the current neural network can be updated by updating the network parameters (e.g., weights, biases, or both) in order to minimize the loss according to the loss function. When the error has been minimized (e.g., by determining whether an error threshold or other stopping criterion has been satisfied), the current neural network and its associated network parameters represent the trained neural network.

In some embodiments, multispectral imaging data and spectral off-resonance data are assembled into training data for training a neural network against the conventional susceptibility weighted images. In some other embodiments, optimal contrast metrics can be developed on the conventional susceptibility weighted images sets, from which a cost function can be constructed and implemented in the training of the neural network architecture. For example, the neural network can be constructed and trained similar to a neural style transfer (“NST”) network, in which the magnitude image(s) from the multispectral imaging data (e.g., T1-weighted image(s), T2-weighted image(s), proton density weighted image(s), combinations thereof) and spectral off-resonance data are input and an image with a susceptibility weighted image contrast is output.

Additionally or alternatively, spectral off-resonance data (e.g., filtered field maps) can be analyzed to identify regions of high off-resonance (e.g., vessels) in both image sets (multispectral imaging data and conventional susceptibility weighted images), and then the contrast between those regions and nearby tissue can be computed. A targeted contrast can be developed on the conventional susceptibility weighted images datasets, and then this contrast computation can be run as a cost function within the neural network architecture on the multispectral imaging data and/or spectral off-resonance data, such that the final output image has the same contrast in those key areas as the conventional susceptibility weighted image.

The one or more trained neural networks are then stored for later use, as indicated at step 406. Storing the neural network(s) may include storing network parameters (e.g., weights, biases, or both), which have been computed or otherwise estimated by training the neural network(s) on the training data. Storing the trained neural network(s) may also include storing the particular network architecture to be implemented. For instance, data pertaining to the layers in the network architecture (e.g., number of layers, type of layers, ordering of layers, connections between layers, hyperparameters for layers) may be stored.

In an example study, images were acquired at 3 Tesla using T1-weighted (TE=8.4 ms, TR=647.2 ms, ETL=12) and T2-weighted (TE=85 ms, TR=3500 ms, ETL=40 and flow-compensation) 3D-MSI acquisitions with 24 spectral bins with 1 kHz spacing. For comparison, conventional SWI imaging was derived from multi-echo GRE images using 3-direction flow compensation (TEₘₑₐₙ=23 ms, TR=37.8 ms, 5 echoes). All images were acquired with 1×1 mm² in-plane resolution across 28 axial 4 mm slice-encoded sections. Acquisition times for the images were five minutes for the 3D-MSI SWI and four minutes for the conventional multi-echo SWI acquisition.

Images were acquired from a single healthy volunteer subject with a large cobalt-chromium acetabular implant component placed on their right forehead region, and then again without the device in place.

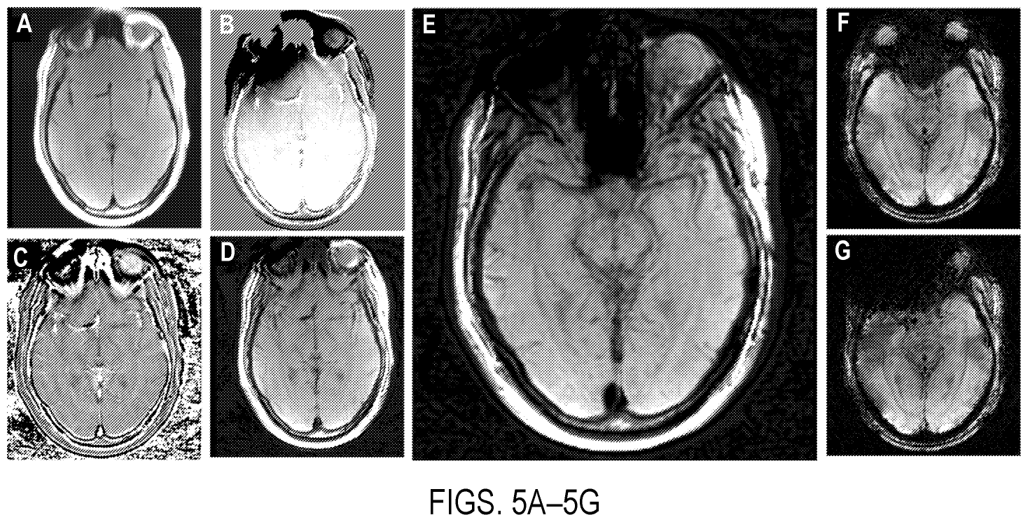

FIGS. 5A-5G shows images that illustrate the 3D-MSI based SWI methods described in the present disclosure. A magnitude 3D-MSI image is shown in FIG. 5A and the spectral off-resonance map derived from the spectral bin data is shown in FIG. 5B. As described above, the spectral off-resonance map is denoised, high-pass filtered (e.g., as shown in FIG. 5C), and then raised to the fourth power. The resulting spectral perturbation map is then multiplied by the magnitude 3D-MSI image (T1-weighted in this case), generating the 3D-MSI SWI concept image shown in FIG. 5D. A minimum intensity projection of this image is shown in FIG. 5E, depicting enhanced visualization of vessels within the brain. For reference, SWI minimum intensity projection images are shown for this slice without (FIG. 5F) and with (FIG. 5G) the metallic implant in place.

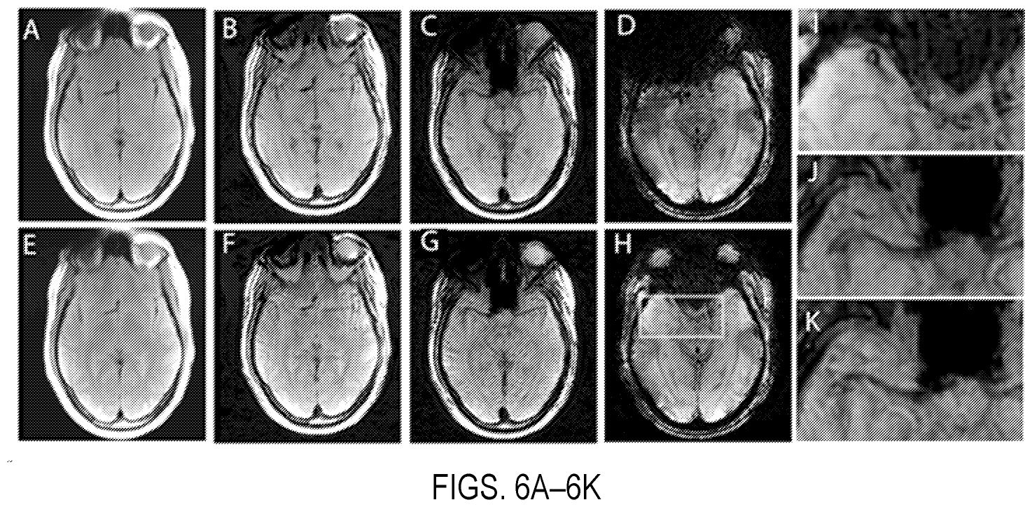

FIGS. 6A-6K show additional examples of 3D-MSI based and conventional SWI based images. FIGS. 6A-6C show T1-weighted images and FIGS. 6E-6G show T2-weighted images. FIGS. 6A and 6E show examples of raw magnitude value images, FIGS. 6B and 6F show examples of SWI-like images derived from the 3D-MSI images, and FIGS. 6C and 6G show examples of minimum intensity projection images generated from the SWI-like images. For reference, conventional SWI images are shown in FIG. 6D with the presence of the implant and in FIG. 6H without the presence of the implant. Zoomed sections show more intricate visualization differences between the approaches for the minimum intensity projection images from the T1-weighted 3D-MSI SWI (FIG. 6I), T2-weighted 3D-MSI SWI (FIG. 6J), and conventional SWI without the implant in place (FIG. 6K).

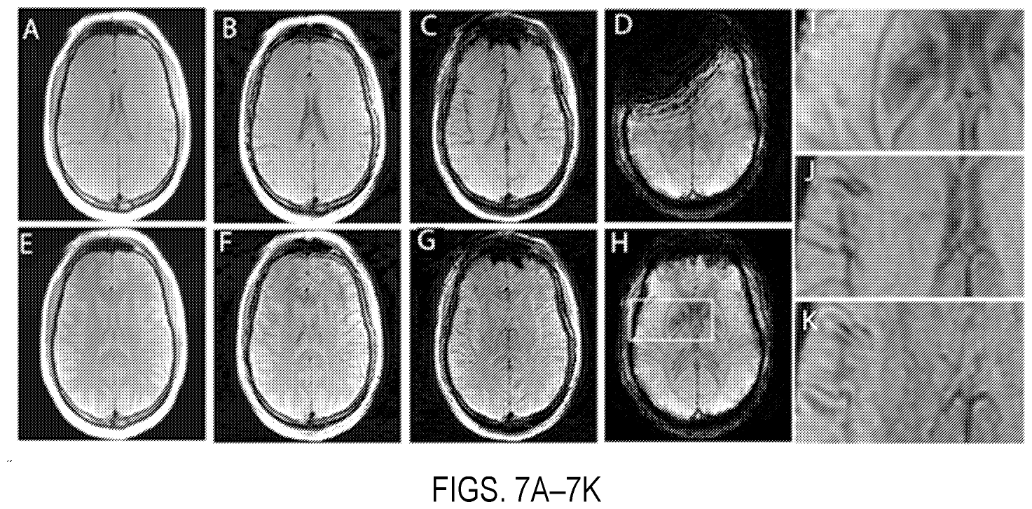

FIGS. 7A-7K show additional examples similar to those shown in FIGS. 6A-6K, but taken from a different slice location within the subject. FIGS. 7A-7C show T1-weighted images and FIGS. 7E-7G show T2-weighted images. FIGS. 7A and 7E show examples of raw magnitude value images, FIGS. 7B and 7F show examples of SWI-like images derived from the 3D-MSI images, and FIGS. 7C and 7G show examples of minimum intensity projection images generated from the SWI-like images. For reference, conventional SWI images are shown in FIG. 7D with the presence of the implant and in FIG. 7H without the presence of the implant. Zoomed sections show more intricate visualization differences between the approaches for the minimum intensity projection images from the T1-weighted 3D-MSI SWI (FIG. 7I), T2-weighted 3D-MSI SWI (FIG. 7J), and conventional SWI without the implant in place (FIG. 7K).

It can be seen from the results in FIGS. 4-7 that the 3D-MSI SWI approach can recover signal that is otherwise lost near metal implants when using conventional SWI approaches. It can also be seen from the zoomed in images of FIGS. 6I-6K and FIGS. 7I-7K that the SWI contrast derived from the 3D-MSI data is variable between the T1-weighted and T2-weighted SWI and the conventional T2*/T1-weighted SWI. While both 3D-MSI approaches can individually enhance the depiction of the vasculature, as expected in an SWI image, and are also likely to detect off-resonance localized variations (e.g., bleeds or calcifications), the two approaches do not have the traditional T2*-weighted background contrast that is typical in SWI images. In some embodiments, a combination of T1-weighted and T2-weighted 3D-MSI images can be used to generate a fused image contrast that is more indicative of the T2*-weighting seen in conventional SWI images.

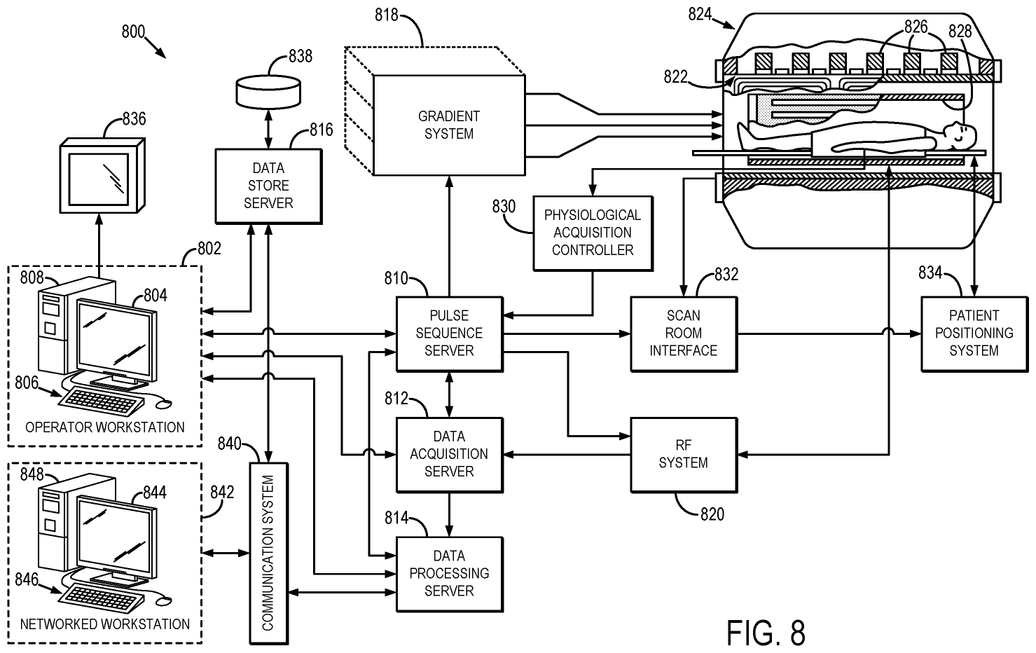

Referring particularly now to FIG. 8, an example of an MRI system 800 that can implement the methods described here is illustrated. The MRI system 800 includes an operator workstation 802 that may include a display 804, one or more input devices 806 (e.g., a keyboard, a mouse), and a processor 808. The processor 808 may include a commercially available programmable machine running a commercially available operating system. The operator workstation 802 provides an operator interface that facilitates entering scan parameters into the MRI system 800. The operator workstation 802 may be coupled to different servers, including, for example, a pulse sequence server 810, a data acquisition server 812, a data processing server 814, and a data store server 816. The operator workstation 802 and the servers 810, 812, 814, and 816 may be connected via a communication system 840, which may include wired or wireless network connections.

The pulse sequence server 810 functions in response to instructions provided by the operator workstation 802 to operate a gradient system 818 and a radiofrequency (“RF”) system 820. Gradient waveforms for performing a prescribed scan are produced and applied to the gradient system 818, which then excites gradient coils in an assembly 822 to produce the magnetic field gradients Gₓ, G_{y}, and G_{z} that are used for spatially encoding magnetic resonance signals. The gradient coil assembly 822 forms part of a magnet assembly 824 that includes a polarizing magnet 826 and a whole-body RF coil 828. The polarizing magnet 826 generates the main magnetic field, B₀, of the MRI system 800. In some implementations, the polarizing magnet 826 may generate a B₀ field with a low-field magnetic field strength (e.g., a magnetic field strength between 0.1-0.3 T), a very-low-field magnetic field strength (e.g., a magnetic field strength between 10 mT-0.1 T), and/or an ultra-low-field magnetic field strength (e.g., a magnetic field strength less than 10 mT). The polarizing magnet 826 may include a superconducting magnet, a permanent magnet, and array of permanent magnets, or other suitable magnet capable of generating a B₀ field.

RF waveforms are applied by the RF system 820 to the RF coil 828, or a separate local coil to perform the prescribed magnetic resonance pulse sequence. Responsive magnetic resonance signals detected by the RF coil 828, or a separate local coil, are received by the RF system 820. The responsive magnetic resonance signals may be amplified, demodulated, filtered, and digitized under direction of commands produced by the pulse sequence server 810. The RF system 820 includes an RF transmitter for producing a wide variety of RF pulses used in MRI pulse sequences. The RF transmitter is responsive to the prescribed scan and direction from the pulse sequence server 810 to produce RF pulses of the desired frequency, phase, and pulse amplitude waveform. The generated RF pulses may be applied to the whole-body RF coil 828 or to one or more local coils or coil arrays.

The RF system 820 also includes one or more RF receiver channels. An RF receiver channel includes an RF preamplifier that amplifies the magnetic resonance signal received by the coil 828 to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

$$M = \sqrt{{I}^{2} + {Q}^{2}} ;$$

and the phase of the received magnetic resonance signal may also be determined according to the following relationship:

$$φ = {tan}^{- 1} ( \frac{Q}{I} ) .$$

The pulse sequence server 810 may receive patient data from a physiological acquisition controller 830. By way of example, the physiological acquisition controller 830 may receive signals from a number of different sensors connected to the patient, including electrocardiograph (“ECG”) signals from electrodes, or respiratory signals from a respiratory bellows or other respiratory monitoring devices. These signals may be used by the pulse sequence server 810 to synchronize, or “gate,” the performance of the scan with the subject's heart beat or respiration.

The pulse sequence server 810 may also connect to a scan room interface circuit 832 that receives signals from various sensors associated with the condition of the patient and the magnet system. Through the scan room interface circuit 832, a patient positioning system 834 can receive commands to move the patient to desired positions during the scan.

The digitized magnetic resonance signal samples produced by the RF system 820 are received by the data acquisition server 812. The data acquisition server 812 operates in response to instructions downloaded from the operator workstation 802 to receive the real-time magnetic resonance data and provide buffer storage, so that data is not lost by data overrun. In some scans, the data acquisition server 812 passes the acquired magnetic resonance data to the data processor server 814. In scans that require information derived from acquired magnetic resonance data to control the further performance of the scan, the data acquisition server 812 may be programmed to produce such information and convey it to the pulse sequence server 810. For example, during pre-scans, magnetic resonance data may be acquired and used to calibrate the pulse sequence performed by the pulse sequence server 810. As another example, navigator signals may be acquired and used to adjust the operating parameters of the RF system 820 or the gradient system 818, or to control the view order in which k-space is sampled. In still another example, the data acquisition server 812 may also process magnetic resonance signals used to detect the arrival of a contrast agent in a magnetic resonance angiography (“MRA”) scan. For example, the data acquisition server 812 may acquire magnetic resonance data and processes it in real-time to produce information that is used to control the scan.

The data processing server 814 receives magnetic resonance data from the data acquisition server 812 and processes the magnetic resonance data in accordance with instructions provided by the operator workstation 802. Such processing may include, for example, reconstructing two-dimensional or three-dimensional images by performing a Fourier transformation of raw k-space data, performing other image reconstruction algorithms (e.g., iterative or backprojection reconstruction algorithms), applying filters to raw k-space data or to reconstructed images, generating functional magnetic resonance images, or calculating motion or flow images.

Images reconstructed by the data processing server 814 are conveyed back to the operator workstation 802 for storage. Real-time images may be stored in a data base memory cache, from which they may be output to operator display 802 or a display 836. Batch mode images or selected real time images may be stored in a host database on disc storage 838. When such images have been reconstructed and transferred to storage, the data processing server 814 may notify the data store server 816 on the operator workstation 802. The operator workstation 802 may be used by an operator to archive the images, produce films, or send the images via a network to other facilities.

The MRI system 800 may also include one or more networked workstations 842. For example, a networked workstation 842 may include a display 844, one or more input devices 846 (e.g., a keyboard, a mouse), and a processor 848. The networked workstation 842 may be located within the same facility as the operator workstation 802, or in a different facility, such as a different healthcare institution or clinic.

The networked workstation 842 may gain remote access to the data processing server 814 or data store server 816 via the communication system 840. Accordingly, multiple networked workstations 842 may have access to the data processing server 814 and the data store server 816. In this manner, magnetic resonance data, reconstructed images, or other data may be exchanged between the data processing server 814 or the data store server 816 and the networked workstations 842, such that the data or images may be remotely processed by a networked workstation 842.

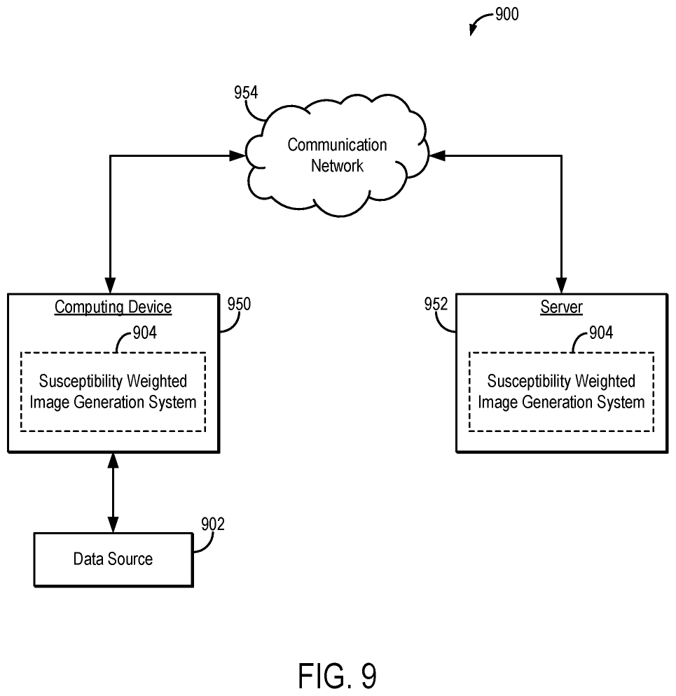

Referring now to FIG. 9, an example of a system 900 for generating susceptibility weighted images from multispectral imaging data in accordance with some embodiments of the systems and methods described in the present disclosure is shown. As shown in FIG. 9, a computing device 950 can receive one or more types of data (e.g., multispectral imaging data, k-space data, magnetic resonance data) from data source 902. In some embodiments, computing device 950 can execute at least a portion of a susceptibility weighted image generation system 904 to generate susceptibility weighted images (or reasonable approximations thereof) from multispectral imaging data received from the data source 902.

Additionally or alternatively, in some embodiments, the computing device 950 can communicate information about data received from the data source 902 to a server 952 over a communication network 954, which can execute at least a portion of the susceptibility weighted image generation system 904. In such embodiments, the server 952 can return information to the computing device 950 (and/or any other suitable computing device) indicative of an output of the susceptibility weighted image generation system 904.

In some embodiments, computing device 950 and/or server 952 can be any suitable computing device or combination of devices, such as a desktop computer, a laptop computer, a smartphone, a tablet computer, a wearable computer, a server computer, a virtual machine being executed by a physical computing device, and so on. The computing device 950 and/or server 952 can also reconstruct images from the data.

In some embodiments, data source 902 can be any suitable source of data (e.g., measurement data, images reconstructed from measurement data, processed image data), such as an MRI system, another computing device (e.g., a server storing measurement data, images reconstructed from measurement data, processed image data), and so on. In some embodiments, data source 902 can be local to computing device 950. For example, data source 902 can be incorporated with computing device 950 (e.g., computing device 950 can be configured as part of a device for measuring, recording, estimating, acquiring, or otherwise collecting or storing data). As another example, data source 902 can be connected to computing device 950 by a cable, a direct wireless link, and so on. Additionally or alternatively, in some embodiments, data source 902 can be located locally and/or remotely from computing device 950, and can communicate data to computing device 950 (and/or server 952) via a communication network (e.g., communication network 954).

In some embodiments, communication network 954 can be any suitable communication network or combination of communication networks. For example, communication network 954 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 954 can be a local area network, a wide area network, a public network (e.g., the Internet), a private or semi-private network (e.g., a corporate or university intranet), any other suitable type of network, or any suitable combination of networks. Communications links shown in FIG. 9 can each be any suitable communications link or combination of communications links, such as wired links, fiber optic links, Wi-Fi links, Bluetooth links, cellular links, and so on.

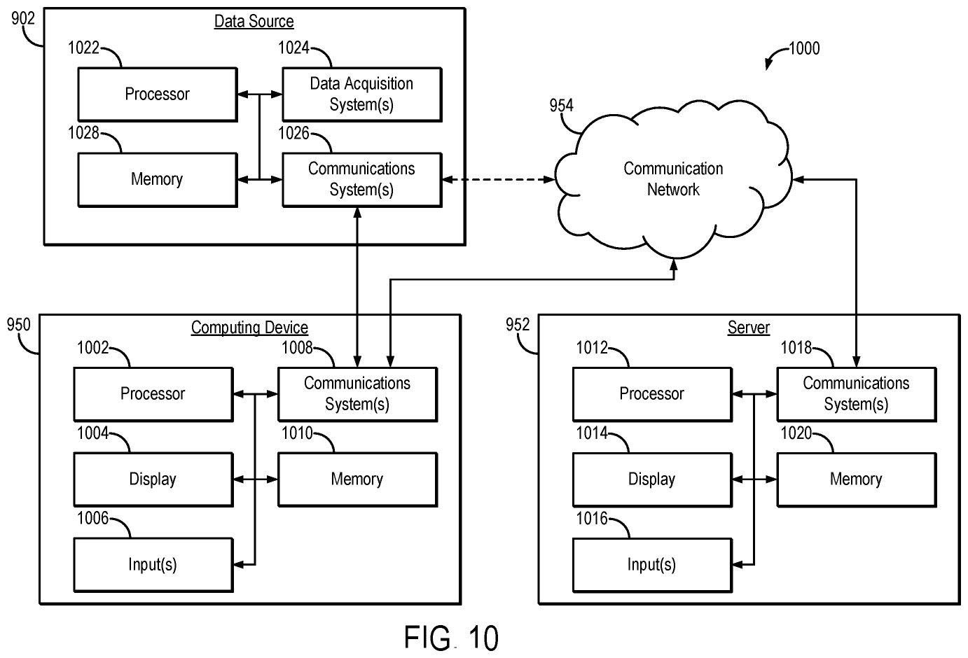

Referring now to FIG. 10, an example of hardware 1000 that can be used to implement data source 902, computing device 950, and server 952 in accordance with some embodiments of the systems and methods described in the present disclosure is shown.

As shown in FIG. 10, in some embodiments, computing device 950 can include a processor 1002, a display 1004, one or more inputs 1006, one or more communication systems 1008, and/or memory 1010. In some embodiments, processor 1002 can be any suitable hardware processor or combination of processors, such as a central processing unit (“CPU”), a graphics processing unit (“GPU”), and so on. In some embodiments, display 1004 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some embodiments, inputs 1006 can include any suitable input devices and/or sensors that can be used to receive user input, such as a keyboard, a mouse, a touchscreen, a microphone, and so on.

In some embodiments, communications systems 1008 can include any suitable hardware, firmware, and/or software for communicating information over communication network 954 and/or any other suitable communication networks. For example, communications systems 1008 can include one or more transceivers, one or more communication chips and/or chip sets, and so on. In a more particular example, communications systems 1008 can include hardware, firmware, and/or software that can be used to establish a Wi-Fi connection, a Bluetooth connection, a cellular connection, an Ethernet connection, and so on.

In some embodiments, memory 1010 can include any suitable storage device or devices that can be used to store instructions, values, data, or the like, that can be used, for example, by processor 1002 to present content using display 1004, to communicate with server 952 via communications system(s) 1008, and so on. Memory 1010 can include any suitable volatile memory, non-volatile memory, storage, or any suitable combination thereof. For example, memory 1010 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some embodiments, memory 1010 can have encoded thereon, or otherwise stored therein, a computer program for controlling operation of computing device 950. In such embodiments, processor 1002 can execute at least a portion of the computer program to present content (e.g., images, user interfaces, graphics, tables), receive content from server 952, transmit information to server 952, and so on. For example, the processor 1002 and the memory 1010 can be configured to perform the methods described herein (e.g., the method of FIG. 1; the method of FIG. 3; the method of FIG. 4).

In some embodiments, server 952 can include a processor 1012, a display 1014, one or more inputs 1016, one or more communications systems 1018, and/or memory 1020. In some embodiments, processor 1012 can be any suitable hardware processor or combination of processors, such as a CPU, a GPU, and so on. In some embodiments, display 1014 can include any suitable display devices, such as an LCD screen, LED display, OLED display, electrophoretic display, a computer monitor, a touchscreen, a television, and so on. In some embodiments, inputs 1016 can include any suitable input devices and/or sensors that can be used to receive user input, such as a keyboard, a mouse, a touchscreen, a microphone, and so on.

In some embodiments, communications systems 1018 can include any suitable hardware, firmware, and/or software for communicating information over communication network 954 and/or any other suitable communication networks. For example, communications systems 1018 can include one or more transceivers, one or more communication chips and/or chip sets, and so on. In a more particular example, communications systems 1018 can include hardware, firmware, and/or software that can be used to establish a Wi-Fi connection, a Bluetooth connection, a cellular connection, an Ethernet connection, and so on.

In some embodiments, memory 1020 can include any suitable storage device or devices that can be used to store instructions, values, data, or the like, that can be used, for example, by processor 1012 to present content using display 1014, to communicate with one or more computing devices 950, and so on. Memory 1020 can include any suitable volatile memory, non-volatile memory, storage, or any suitable combination thereof. For example, memory 1020 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1020 can have encoded thereon a server program for controlling operation of server 952. In such embodiments, processor 1012 can execute at least a portion of the server program to transmit information and/or content (e.g., data, images, a user interface) to one or more computing devices 950, receive information and/or content from one or more computing devices 950, receive instructions from one or more devices (e.g., a personal computer, a laptop computer, a tablet computer, a smartphone), and so on.

In some embodiments, the server 952 is configured to perform the methods described in the present disclosure. For example, the processor 1012 and memory 1020 can be configured to perform the methods described herein (e.g., the method of FIG. 1; the method of FIG. 3; the method of FIG. 4).

In some embodiments, data source 902 can include a processor 1022, one or more data acquisition systems 1024, one or more communications systems 1026, and/or memory 1028. In some embodiments, processor 1022 can be any suitable hardware processor or combination of processors, such as a CPU, a GPU, and so on. In some embodiments, the one or more data acquisition systems 1024 are generally configured to acquire data, images, or both, and can include an MRI system. Additionally or alternatively, in some embodiments, the one or more data acquisition systems 1024 can include any suitable hardware, firmware, and/or software for coupling to and/or controlling operations of an MRI system. In some embodiments, one or more portions of the data acquisition system(s) 1024 can be removable and/or replaceable.

Note that, although not shown, data source 902 can include any suitable inputs and/or outputs. For example, data source 902 can include input devices and/or sensors that can be used to receive user input, such as a keyboard, a mouse, a touchscreen, a microphone, a trackpad, a trackball, and so on. As another example, data source 902 can include any suitable display devices, such as an LCD screen, an LED display, an OLED display, an electrophoretic display, a computer monitor, a touchscreen, a television, etc., one or more speakers, and so on.

In some embodiments, communications systems 1026 can include any suitable hardware, firmware, and/or software for communicating information to computing device 950 (and, in some embodiments, over communication network 954 and/or any other suitable communication networks). For example, communications systems 1026 can include one or more transceivers, one or more communication chips and/or chip sets, and so on. In a more particular example, communications systems 1026 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 1028 can include any suitable storage device or devices that can be used to store instructions, values, data, or the like, that can be used, for example, by processor 1022 to control the one or more data acquisition systems 1024, and/or receive data from the one or more data acquisition systems 1024; to generate images from data; present content (e.g., data, images, a user interface) using a display; communicate with one or more computing devices 950; and so on. Memory 1028 can include any suitable volatile memory, non-volatile memory, storage, or any suitable combination thereof. For example, memory 1028 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1028 can have encoded thereon, or otherwise stored therein, a program for controlling operation of medical image data source 902. In such embodiments, processor 1022 can execute at least a portion of the program to generate images, transmit information and/or content (e.g., data, images, a user interface) to one or more computing devices 950, receive information and/or content from one or more computing devices 950, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the invention.

1. A method for magnetic resonance imaging, the method comprising:
(a) accessing multispectral imaging data with a computer system, wherein the multispectral imaging data comprise magnetic resonance data acquired using multiple different spectral offsets;
(b) generating a spectral off-resonance map from the multispectral imaging data using the computer system; and
(c) generating an image with a susceptibility weighted image contrast by multiplying a magnitude image from the multispectral imaging data with the spectral off-resonance map.

2. The method of claim 1, wherein generating the spectral off-resonance map comprises generating spectral bin data from the multispectral imaging data and estimating the spectral off-resonance map from the spectral bin data.

3. The method of claim 1, wherein generating the spectral off-resonance map further includes denoising the spectral off-resonance map, filtering the spectral off-resonance map, and multiplying the spectral off-resonance map by a function.

4. The method of claim 3, wherein filtering the spectral off-resonance map comprises high-pass filtering the spectral off-resonance map.

5. The method of claim 3, wherein multiplying the spectral off-resonance map by the function comprises multiplying the spectral off-resonance map by a power function.

6. The method of claim 5, wherein the power function is a fourth power function such that multiplying the spectral off-resonance map with the fourth power function comprises raising values in the spectral off-resonance map to the fourth power.

7. The method of claim 1, wherein accessing the multispectral imaging data comprises acquiring the multispectral imaging data with a magnetic resonance imaging (MRI) system.

8. The method of claim 7, wherein the multispectral imaging data are acquired using a three-dimensional multispectral imaging (3D-MSI) pulse sequence.

9. The method of claim 7, wherein the MRI system is a low-field MRI system having a B field with a magnetic field strength less than 0.3 T.

10. The method of claim 9, wherein the B₀ field of the low-field MRI system has a magnetic field strength between 0.1 T and 0.3 T.

11. The method of claim 9, wherein the B₀ field of the low-field MRI system has a magnetic field strength between 10 mT and 0.1 T.

12. The method of claim 9, wherein the B₀ field of the low-field MRI system has a magnetic field strength less than 10 mT.

13. The method of claim 1, wherein the multispectral imaging data comprise at least one of T1-weighted images acquired with T1-weighting or T2-weighted images acquired with T2-weighting.

14. The method of claim 13, further comprising combining the T1-weighted images and the T2-weighted images to generate an image with a composite image contrast, and wherein the image with the composite image contrast is the magnitude image multiplied by the spectral off-resonance map to generate the image with the susceptibility weighted image contrast.

15. The method of claim 1, wherein the multispectral imaging data were acquired from a subject using a magnetic resonance imaging (MRI) system while a metal object was located within a bore of the MRI system, and wherein the image with the susceptibility weighted image contrast has reduced image artifacts attributable to the metal object.

16. The method of claim 15, wherein the metal object is a surgical implant.

17. A method for magnetic resonance imaging, the method comprising:
(a) accessing multispectral imaging data with a computer system, wherein the multispectral imaging data comprise magnetic resonance data acquired using multiple different spectral offsets;
(b) accessing spectral off-resonance data with the computer system, wherein the spectral off-resonance data are generated from the multispectral imaging data;
(c) accessing a neural network with the computer system, wherein the neural network has been trained on training data to generate images with susceptibility weighted image contrast based on inputs of multispectral imaging data and spectral off-resonance data; and
(d) generating an image with a susceptibility weighted image contrast by inputting the multispectral imaging data and the spectral off-resonance data to the neural network using the computer system, generating output as the image with the susceptibility weighted image contrast.

18. The method of claim 17, wherein the neural network comprises a convolutional neural network.

19. The method of claim 18, wherein the convolutional neural network implements a V-Net architecture.

20. The method of claim 17, wherein accessing the multispectral imaging data comprises acquiring the multispectral imaging data with a magnetic resonance imaging (MRI) system.

21. The method of claim 20, wherein the multispectral imaging data are acquired using a three-dimensional multispectral imaging (3D-MSI) pulse sequence.

22. The method of claim 20, wherein the MRI system is a low-field MRI system having a B₀ field with a magnetic field strength less than 0.3 T.

23. The method of claim 22, wherein the B₀ field of the low-field MRI system has a magnetic field strength between 0.1 T and 0.3 T.

24. The method of claim 22, wherein the B₀ field of the low-field MRI system has a magnetic field strength between 10 mT and 0.1 T.

25. The method of claim 22, wherein the B₀ field of the low-field MRI system has a magnetic field strength less than 10 mT.
